(19) United States
(12) Patent Application Publication
(10) Pub. No.: US 2018/0140804 A1
(43) Pub. Date: May 24, 2018

(54) BALLOON CATHETER

(71) Applicant: ASAHI INTECC CO., LTD., Nagoya-shi (JP)

(72) Inventors: Toshihiko TSUKAMOTO, Owariasahi-shi (JP); Keisuke KAWAGUCHI, Seto-shi (JP); Nobuyoshi YAMANAKA, Owariasahi-shi (JP); Yoshiki KANEKO, Nishio-shi (JP)

(73) Assignee: ASAHI INTECC CO., LTD., Nagoya-shi (JP)

(21) Appl. No.: 15/622,622

(22) Filed: Jun. 14, 2017

Related U.S. Application Data
(63) Continuation of application No. PCT/JP2016/084550, filed on Nov. 22, 2016.

(51) Int. Cl. A61M 25/10; A61B 17/12; A61M 29/02

(52) U.S. Cl. CPC A61M 25/1002; A61B 17/12136; A61M 2025/1086; A61M 2025/1004; A61M 2025/1075; A61M 29/02

(57) ABSTRACT

A balloon catheter includes a balloon. The balloon allows restraint-free arrangement of an irregular region on the balloon for quickly inserting the balloon into a stenosis or stricture or an obstructed part while the balloon is in a contracted state, and allows for securely fixing the balloon within the stenosis or stricture or the obstructed part while the balloon is in an expanded state. The balloon can be easily produced, and can be folded without restraint (without requiring differentiation between the irregular region and the rest of the balloon). The balloon includes a base portion having an irregular region on its surface, and a coating agent coating the surface of the base portion. When the balloon is not expanded, the surface of the balloon is smooth and flat; and while the balloon is in an expanded state, the surface of the balloon is irregular.

### CROSS REFERENCE TO RELATED APPLICATION

This application is a continuation of PCT/JP2016/0084550 filed on Nov. 22, 2016, the contents of which are incorporated by reference herein in their entirety.

### BACKGROUND

The disclosed embodiments relate to a medical device. Specifically, the disclosed embodiments relate to a balloon catheter for insertion into a stenosis or stricture or an obstructed part (an affected area) that is formed within an internal lumen such as a blood vessel, a bile duct, or a pancreatic duct for the purpose of dilating the affected area, thereby ensuring the flow of blood, bile (biliary fluid), pancreatic juice, or the like.

A stenosis or stricture or an obstructed part that is formed within an internal lumen such as a blood vessel, a bile duct, or a pancreatic duct interrupts the flow of blood, bile (biliary fluid), pancreatic juice, or the like. Conventionally, there are methods widely used for treating the affected area, which use a balloon catheter.

A conventional balloon catheter primarily comprises an expandable balloon, an outer tube fixed to a proximal end of the balloon, and an inner tube accommodated within the interior of both the balloon and the outer tube. A guidewire is inserted into the inner tube and then extends through the inner tube. An expansion lumen is interposed between the outer tube and the inner tube, and through this expansion lumen, liquid (such as a contrast medium or physiological saline) is introduced to expand the balloon.

In a typical balloon catheter, the balloon is folded around the outer circumference of the inner tube while the catheter is being inserted into an internal lumen, and then when the catheter reaches the affected area, the balloon is expanded within the affected area.

During insertion of the catheter with the balloon folded around the outer circumference of the inner tube, the frictional force between the balloon and the internal lumen is preferably as small as possible for quick insertion of the balloon into the affected area.

While the balloon is in an expanded state within the affected area, the frictional force between the balloon and the affected area is preferably as great as possible for securely fixing the balloon within the affected area.

For example, Japanese Patent Application Publication No. 2005-224635 (JP 2005-224635 A) describes a balloon catheter that has a low coefficient of sliding friction while the balloon is in a contracted state, and a high coefficient of sliding friction while the balloon is in an expanded state.

More specifically, this balloon catheter has the following configuration: while the balloon is in a contracted state, a region with a low coefficient of friction stretches over the entire surface of the balloon, and a region with a high coefficient of friction is enclosed within the folds of the balloon; and while the balloon is in an expanded state, the region with a high coefficient of friction comes out of the folds of the balloon to stretch over the surface of the balloon, allowing fixation of the balloon within the affected area.

However, the balloon catheter described in JP 2005-224635 A requires a complicated balloon-producing process in order to (i) provide the region with a low coefficient of friction and the region with a high coefficient of friction in an arrangement in which these two regions appear alternately about the circumference of the balloon when viewed in cross section from a longitudinal axis of the balloon, and (ii) have the region with a low coefficient of friction stretch over the entire surface of the balloon and enclose the region with a high coefficient of friction within the folds of the balloon while the balloon is in a contracted state.

The balloon catheter described in JP 2005-224635 A has another problem: because the region with a low coefficient of friction and the region with a high coefficient of friction need to be alternately arranged about the circumference of the balloon when viewed in cross section as described above, there is limited ability to vary the arrangement of the region with a low coefficient of friction and the region with a high coefficient of friction.

### SUMMARY

The disclosed embodiments have been devised based on the above circumstances. An object of the disclosed embodiments is to provide a balloon catheter comprising a balloon. The balloon allows restraint-free arrangement of a region with a high coefficient of friction (hereinafter, also called an irregular region) on the balloon that allows both for quickly inserting the balloon into a stenosis or stricture or an obstructed part (affected area) while the balloon is in a contracted state, and for securely fixing the balloon within the affected area while the balloon is in an expanded state. It also can be easily produced, and can be folded without restraint (namely without requiring differentiation between the irregular region and the rest of the balloon).

In order to achieve the object above, the disclosed embodiments include a balloon catheter comprising a balloon. The balloon comprises a base layer having an irregular region on a surface thereof; and a coating layer coating the surface of the base layer. Before the balloon is expanded, the surface of the balloon is smooth and flat due to the presence of the coating layer, and while the balloon is in an expanded state, the surface of the balloon is irregular due to the presence of the irregular region of the base layer. With this configuration, the balloon allows restraint-free arrangement of the irregular region on the balloon and can be easily produced. In addition, the balloon can be folded without restraint, namely without requiring differentiation between the irregular region and the rest of the balloon.

The irregular region of the base layer may stretch over the entire circumference of the balloon when viewed in cross section from the longitudinal direction. With this configuration, the balloon can be easily fixed to an affected area regardless of where the affected area is located.

Upon expansion of the balloon, the surface of the coating layer can break, causing the surface of the balloon to become irregular. Because of this configuration, the balloon can be even more easily fixed to an affected area.

### BRIEF DESCRIPTION OF THE DRAWINGS

FIG. 1 is a schematic, overall view of a balloon catheter according to the disclosed embodiments (the balloon in the figure is in a contracted state).

FIG. 2 is a sectional view of the balloon in FIG. 1 in a slightly expanded state, taken from line A-A.

FIG. 3 is a sectional view of the balloon in FIG. 1 that is folded differently and slightly expanded, taken from line A-A.

FIG. 4 is a schematic, overall view of the balloon catheter in FIG. 1 (the balloon in this figure is in an expanded state).

FIG. 5 illustrates an irregular region of the balloon catheter in FIG. 1 (the balloon in this figure is in an expanded state).

FIG. 6 is a sectional view taken from line B-B of FIG. 5.

FIG. 7 illustrates an irregular region of a balloon catheter according to the disclosed embodiments (the balloon in this figure is in an expanded state).

FIG. 8 is a sectional view taken from line C-C of FIG. 7.

FIG. 9 illustrates an irregular region of a balloon catheter according to the disclosed embodiments (the balloon in this figure is in an expanded state).

FIG. 10 is a sectional view taken from line D-D of FIG. 9.

### DETAILED DESCRIPTION OF EMBODIMENTS

Embodiments of the present invention will be described with reference to drawings.

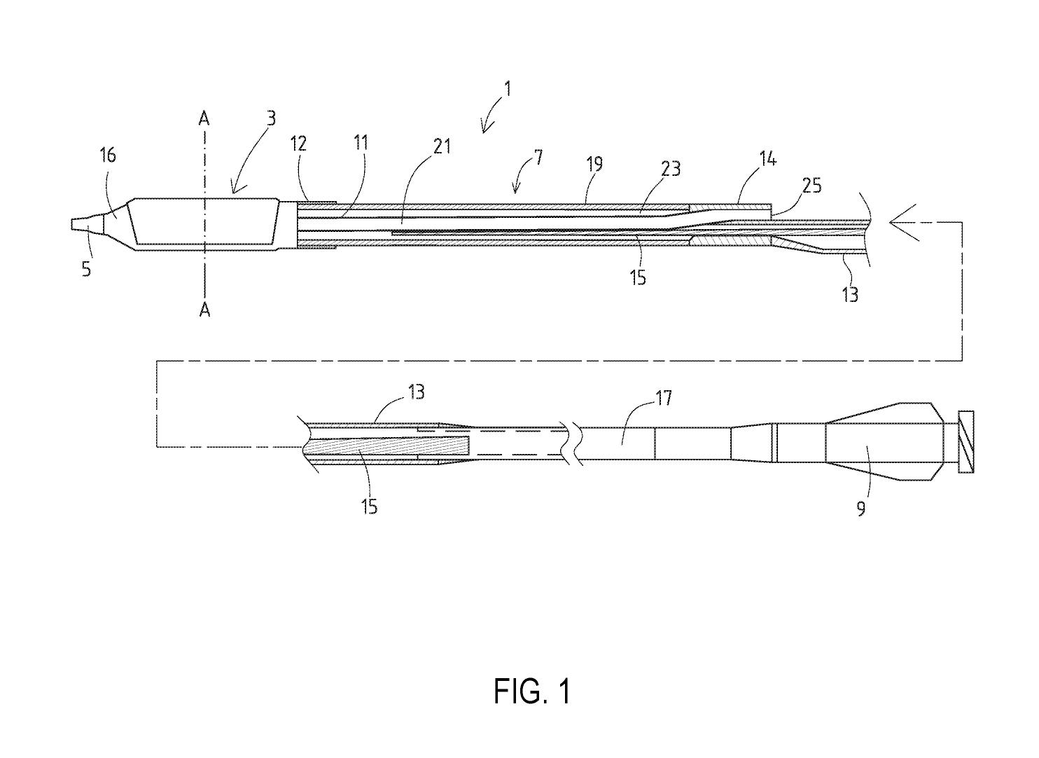

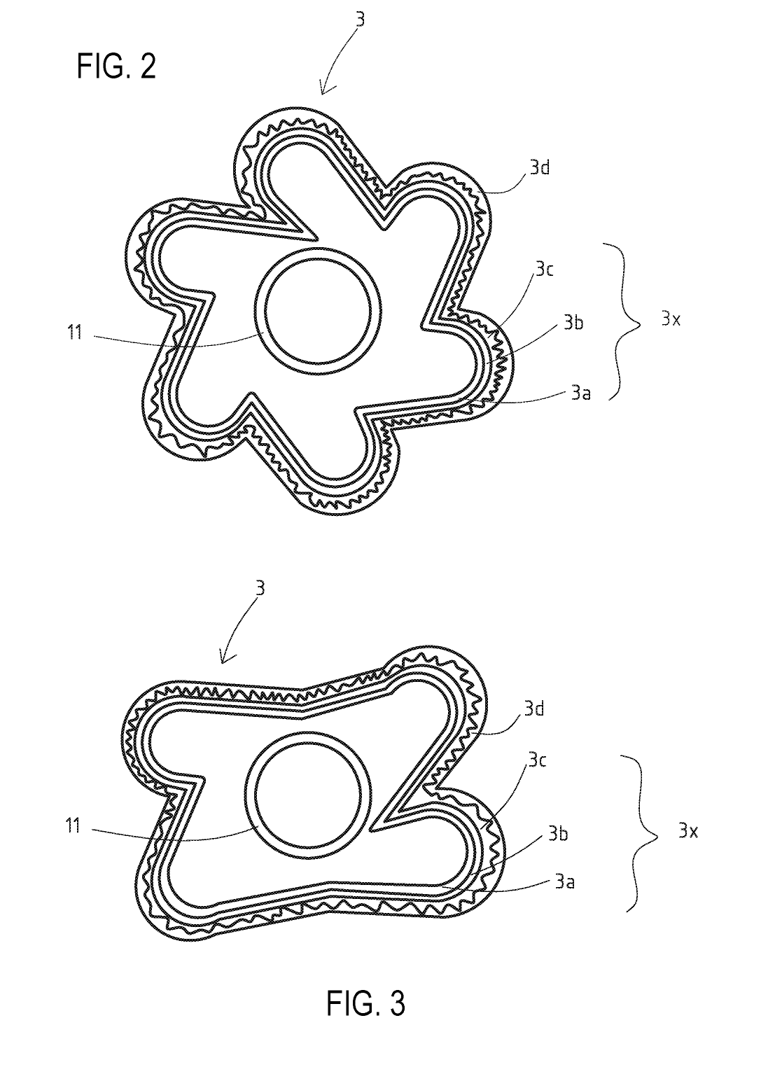

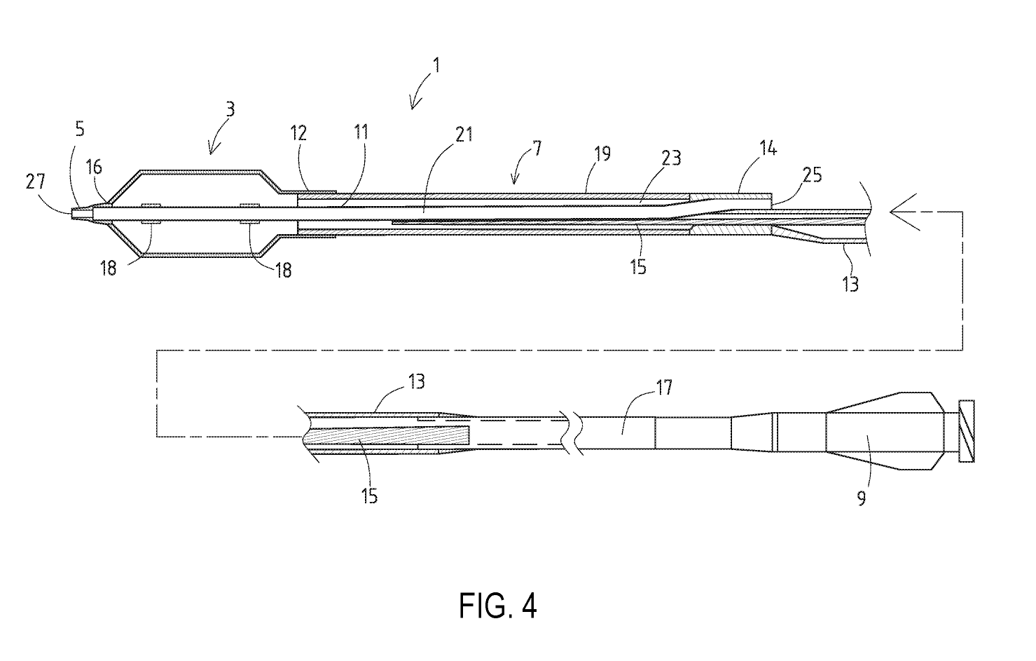

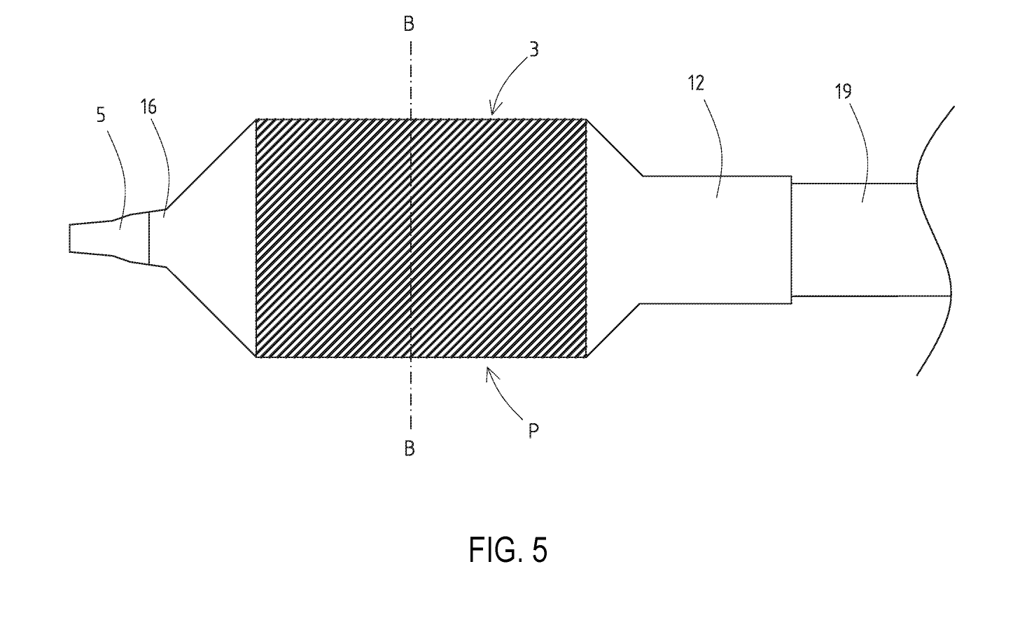

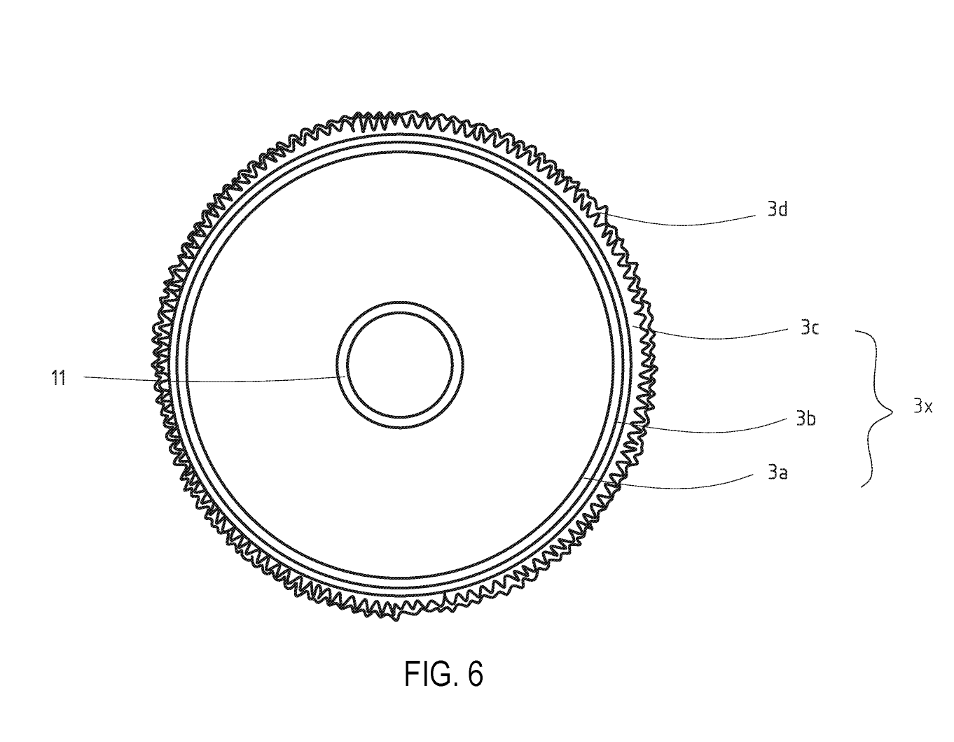

FIG. 1 is a schematic, overall view of a balloon catheter according to the disclosed embodiments (the balloon in the figure is in a contracted state); FIG. 2 is a sectional view of the balloon in FIG. 1 in a slightly expanded state, taken from line A-A; FIG. 3 is a sectional view of the balloon in FIG. 1 that is folded differently and slightly expanded, taken from line A-A; FIG. 4 is a schematic, overall view of the balloon catheter in FIG. 1 (the balloon in the figure is in an expanded state); FIG. 5 illustrates an irregular region of the balloon catheter in FIG. 1 (the balloon in the figure is in an expanded state); and FIG. 6 is a sectional view taken from line B-B of FIG. 5.

A balloon catheter 1 in FIG. 1 is used, for example, for treating (dilating) a stenosis or stricture or an obstructed part (an affected area) that is formed within an internal lumen such as a blood vessel, a bile duct, or a pancreatic duct. The balloon catheter 1 comprises a balloon 3, an outer tube 7, a connector 9, an inner tube 11, and a distal-end tip 5. The balloon 3 in FIG. 1 is in a contracted state.

The balloon 3 may be formed of resin. A distal-end-fitting member 16 of the balloon 3 is fixed to a distal end of the inner tube 11 and to the distal-end tip 5. A proximal-end-fitting member 12 of the balloon 3 is fixed to a distal end of the outer tube 7.

The distal-end-fitting member 16 of the balloon 3 as shown in FIG. 4 is fixed in an arrangement in which it covers the distal-end tip 5 and the distal end of the inner tube 11. Alternatively, the distal-end-fitting member 16 of the balloon 3 may be fixed and interposed between the distal end of the inner tube 11 and the distal-end tip 5.

The proximal-end-fitting member 12 of the balloon 3 as shown in FIG. 4 is fixed to an outer circumference of the distal end of the outer tube 7. Alternatively, the proximal-end-fitting member 12 of the balloon 3 may be fixed to an inner circumference of the distal end of the outer tube 7.

The outer tube 7 is a tubular component constituting an expansion lumen 23 that feeds liquid (such as a contrast medium or physiological saline) for expanding the balloon 3. The outer tube 7 is composed of, from the distal-end side, an outer-tube distal end 19, a guidewire port 14, an outer-tube middle portion 13, and an outer-tube proximal end 17. Each of the outer-tube distal end 19 and the outer-tube middle portion 13 may be made of a resin such as a polyamide, a polyamide elastomer, a polyolefin, a polyester, or a polyester elastomer. The outer-tube distal end 19, the outer-tube middle portion 13, and the inner tube 11 are fixed to each other at the guidewire port 14.

The outer-tube distal end 19 accommodates the inner tube 11, which is inserted thereinto. The expansion lumen 23 described above is disposed between the outer-tube distal end 19 and the inner tube 11.

The outer-tube proximal end 17 is a tubular metal component, called a hypotube. A distal end of the outer-tube proximal end 17 is accommodated within and fixed to a proximal end of the outer-tube middle portion 13. A proximal end of the outer-tube proximal end 17 has the connector 9 attached thereto. A liquid for expanding the balloon 3 (such as a contrast medium or physiological saline) is fed from an indeflator (not shown, attachable to the connector 9) into the expansion lumen 23 and travels to the balloon 3 to expand it.

The material of the outer-tube proximal end 17 is not particularly limited and may be a super-elastic alloy such as stainless steel (SUS304) or Ni—Ti alloy.

Accommodated in the interior of the inner tube 11 is a guidewire lumen 21 into which a guidewire is inserted. A proximal end of the inner tube 11 is fixed to the guidewire port 14 of the outer tube 7 to form a proximal-end-side guidewire port 25.

The distal end of the inner tube 11 is fixed to the distal-end tip 5 and to the distal-end-fitting member 16 of the balloon 3. The distal-end tip 5 is a tapered component having an outer diameter that gradually decreases toward its distal end, and may be made of a flexible resin. The resin is not particularly limited and may be a polyurethane or a polyurethane elastomer, for example.

The distal-end tip 5 is a tubular component fixed to the distal end of the guidewire lumen 21 and having a distal-end-side guidewire port 27 on its distal end (see FIG. 4).

So as to enable tracking of the location of the balloon 3 under radiation, the inner tube 11 may comprise two radiopaque markers 18 (see FIG. 4) in the interior of the balloon 3.

A reinforcing member 15 is attached to the inner circumference of the distal end of the outer-tube proximal end 17. The reinforcing member 15 is a tapered metal wire that has a circular cross section tapered toward its distal end. The material of the reinforcing member 15 is not particularly limited and may be a super-elastic alloy such as stainless steel (SUS304) or Ni—Ti alloy.

The reinforcing member 15 extends from the distal end of the outer-tube proximal end 17 through the outer-tube middle portion 13 and then through the proximal-end-side guidewire port 25 to reach near the distal end of the outer-tube distal end 19. As shown, the distal end of the reinforcing member 15 is not fixed to either the outer tube 7 or the inner tube 11. Alternatively, the distal end of the reinforcing member 15 may be fixed and interposed between the outer tube 7 and the inner tube 11.

Next, the balloon 3 is described in detail.

The balloon 3 in FIG. 2 is slightly expanded from a state in which it is folded at six locations along its circumference when viewed in cross section from the longitudinal direction of the balloon catheter 1.

As shown in FIG. 2, the balloon 3 comprises a base portion 3x and a coating agent 3d such as polyvinylpyrrolidone (PVP) or hyaluronic acid coating a surface of the base portion 3x to form a coating layer. The base portion has a three-layer structure including a first base layer 3a made of a thermoplastic polyamide elastomer or the like, a second base layer 3b overlaid on the surface of the first base layer 3a and made of another thermoplastic polyamide elastomer or the like, and a third base layer 3c overlaid on the surface of the second base layer 3b and made of a polyamide resin or the like.

As shown in FIG. 2, the entire surface of the base portion 3x of the balloon 3 has an irregular texture (has an uneven surface morphology). Namely, the entire circumference of the base portion 3x of the balloon 3 when viewed in cross section from the longitudinal direction of the balloon catheter 1 is irregular. Before the balloon 3 is expanded, however, the surface of the balloon 3 with the coating agent 3d coating the base portion 3x is smooth and flat (not textured).

In the base portion 3x shown in FIG. 2, the surfaces of the first base layer 3a and the second base layer 3b are smooth and flat and the surface of the third base layer 3c is irregular (has an irregular texture). However, the balloon 3 is not limited to this configuration. As long as the surface of the base portion 3x is irregular, the second base layer 3b and the third base layer 3c may be smooth and flat with the first base layer 3a being irregular, or all of the first base layer 3a, the second base layer 3b, and the third base layer 3c may be irregular. In order to make the surface of the balloon 3 markedly irregular while the balloon 3 is in an expanded state (as described below), however, it is preferable that at least the third base layer 3c is irregular because the third base layer 3c is adjacent to the bottom side of the coating agent 3d.

The irregular profile of the base layers can be formed by various methods that are typically employed for resin film production, such as surface coating (e.g. coating), physical roughening treatment (e.g. sand-matting), and electrical discharge treatment.

The base portion 3x has been described as having a three-layer structure including the first base layer 3a, the second base layer 3b, and the third base layer 3c. Alternatively, the base portion 3x may have a two-layer structure or a one-layer structure. In consideration of the pressure resistance of the balloon 3 as well as the inflated (expanded) state and the deflated (contracted) state of the balloon 3, however, a three-layer structure is preferable.

Although the entire circumference of the base portion 3x of the balloon 3 when viewed in cross section from the longitudinal direction of the balloon catheter 1 is irregular, the surface of the balloon 3 with the coating agent 3d coating the base portion 3x is smooth and flat. Therefore, the balloon 3 can be folded without restraint.

For example, the balloon 3 in FIG. 3 (folded differently from the one in FIG. 2) is folded at four locations along its circumference when viewed in cross section from the longitudinal direction of the balloon catheter 1, in a slightly expanded state.

The balloon 3 has been described as being folded at four or six locations along its circumference when viewed in cross section from the longitudinal direction of the balloon catheter 1 as shown in FIG. 2 and FIG. 3. However, the balloon 3 is not limited to this configuration. The balloon 3 may be folded in any other configuration without restraint.

Next, the balloon 3 in an expanded state is described below.

When the balloon 3 is expanded, an irregular region P appears on the surface of the balloon 3 as shown in FIG. 5. In this embodiment, the irregular region P appears only where the balloon 3 has expanded to its maximum diameter. Alternatively, when it is preferable for production of the balloon 3 to have the irregular region stretch over the entire balloon surface, the irregular region P may be present over the distal-end-fitting member 16 of the balloon 3 and/or the proximal-end-fitting member 12 of the balloon 3 as well.

The balloon 3 comprises the base portion 3x including the irregular third base layer 3c overlaid on the smooth and flat surfaces of the first base layer 3a and the second base layer 3b, and the coating agent 3d coating the surface of the base portion 3x, as described above. When the balloon 3 is expanded, the surface of the balloon 3 becomes irregular together with the coating agent 3d due to the presence of the irregular region of the base portion 3x, as shown in FIG. 6.

As shown, the irregular region P is present where the balloon 3 has expanded to its maximum diameter. Alternatively, the irregular region P may extend beyond where the balloon 3 has expanded to its maximum diameter to include the surface of the distal-end-fitting member 16 of the balloon 3 and/or the proximal-end-fitting member 12 of the balloon 3 as described above, or may in general stretch over any desired area.

The irregular region P may have any surface profile without restraint.

Thus, the balloon catheter 1 according to this embodiment comprises the balloon 3, and the balloon comprises the base portion 3x having an irregular surface and the coating agent 3d coating the surface of the base portion 3x, with the following configuration: before the balloon 3 is expanded, the surface of the balloon 3 is smooth and flat due to the presence of the coating agent 3d; and while the balloon 3 is in an expanded state, the surface of the balloon 3 is irregular due to the irregular surface of the base portion 3x, which impinges on and deforms the coating agent 3d so that the surface of the coating agent 3d (and thus the corresponding surface of the balloon 3) also becomes irregular. This configuration allows for restraint-free arrangement of the irregular region P on the balloon 3 for quickly inserting the balloon 3 into a stenosis or stricture or an obstructed part while the balloon 3 is in a contracted state, and allows for securely fixing the balloon 3 within the stenosis or stricture or the obstructed part while the balloon 3 is in an expanded state.

In the case that the irregular region P stretches over the entire circumference of the balloon when viewed in cross section from the longitudinal direction of the balloon catheter 1, the balloon 3 can be easily fixed to an affected area regardless of where the affected area is located. In addition, the balloon 3 can be easily produced and can be folded without restraint (without requiring differentiation between the irregular region P and the rest of the balloon).

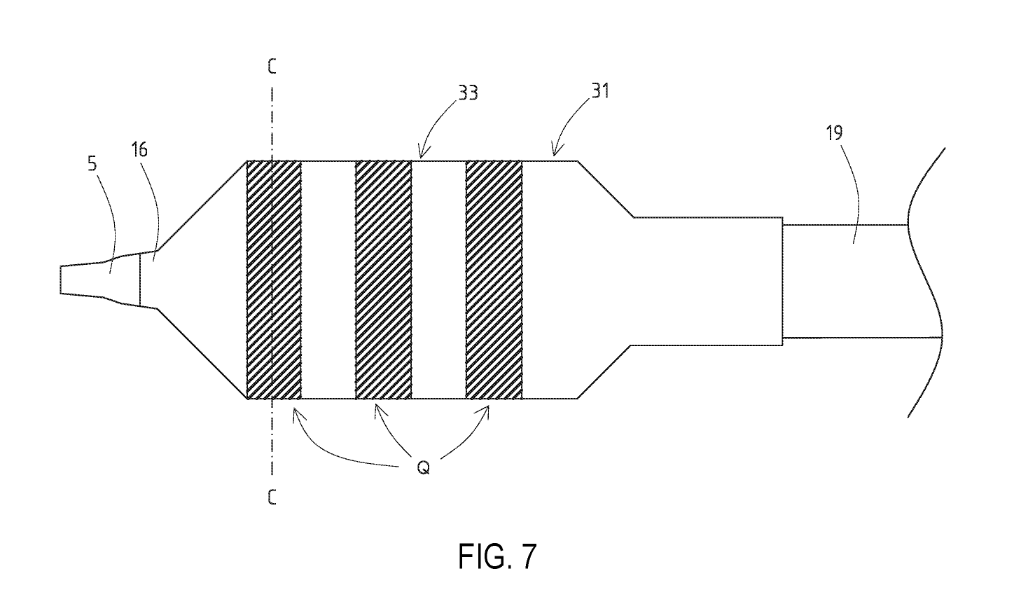

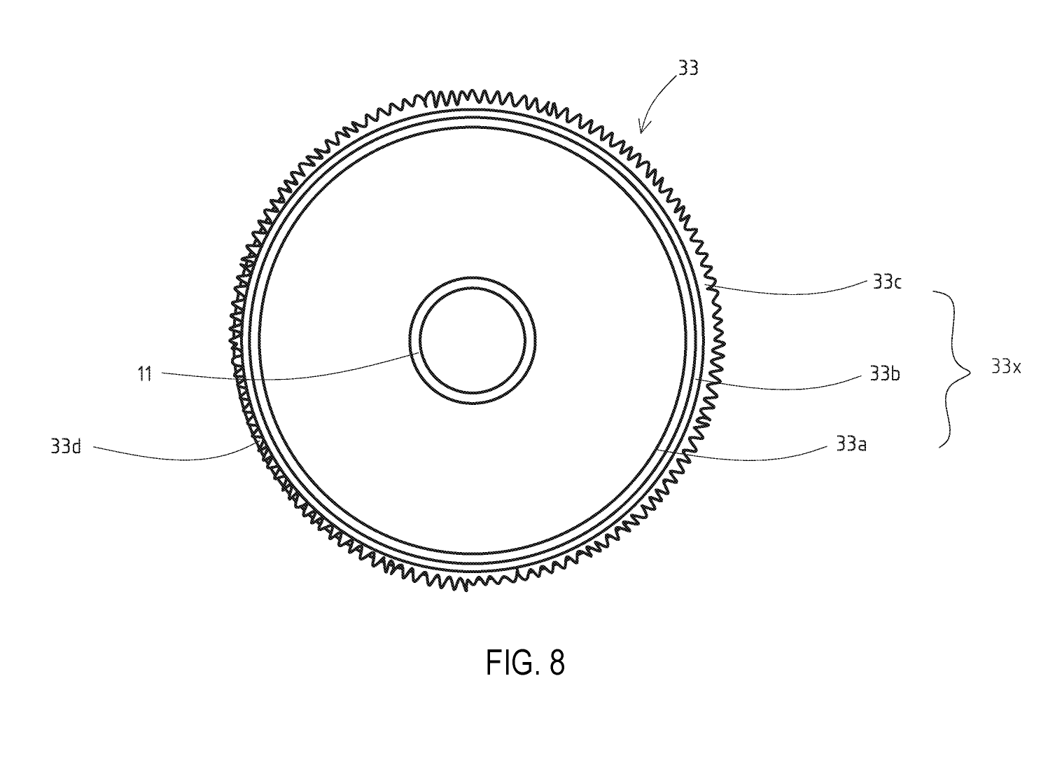

FIG. 7 illustrates an irregular region of a balloon catheter 31 according to the disclosed embodiments (the balloon in the figure is in an expanded state), and FIG. 8 is a sectional view taken from line C-C of FIG. 7.

The following description only includes the differences from the balloon catheter 1.

Referring to FIG. 7, the balloon catheter 31 is substantially the same as the balloon catheter 1 except for the balloon.

An irregular region Q of a balloon 33 is different from the irregular region P of the balloon 3. More specifically, the irregular region P of the balloon 3 stretches over the entire circumference of the balloon 3 when viewed in cross section and stretches in the longitudinal direction of the balloon catheter 1 over the entire area where the balloon 3 has expanded to its maximum diameter. The irregular region Q of the balloon 33 stretches over the entire circumference of the balloon 33 when viewed in cross section as shown in FIG. 8, but is formed at multiple regions (three regions as shown in FIG. 7) regularly spaced in the longitudinal direction of the balloon catheter 31.

As discussed above, the balloon 3 comprises the base portion 3x including the irregular third base layer 3c overlaid on the smooth and flat surfaces of the first base layer 3a and the second base layer 3b, and the coating agent 3d coating the surface of the base portion 3x, with the following configuration: before the balloon 3 is expanded, the surface of the balloon 3 is smooth and flat due to the presence of the coating agent 3d; and when the balloon 3 is expanded, the irregular surface of the base portion 3x raises the coating agent 3d, and thereby the surface of the balloon 3 becomes irregular in the irregular region P.

On the other hand, the balloon 33 comprises a base portion 33x constituted of an irregular third base layer 33c (with a partly irregular surface) overlaid on the smooth and flat surfaces of a first base layer 33a and a second base layer 33b, and a coating agent 33d coating the surface of the base portion 33x, with the following configuration: before the balloon 33 is expanded, the surface of the balloon 33 is smooth and flat due to the presence of the coating agent 33d (as in FIG. 2 and FIG. 3); and when the balloon 33 is expanded, the irregular portion of the surface of the base portion 33x punctures and protrudes through the surface of the coating agent 33d, and thereby the surface of the balloon 33 becomes irregular in the irregular region Q.

The balloon 33 in a contracted state is not directly described but is the same as the corresponding state for the balloon 3. The balloon 33 can be folded at four or six locations along its circumference when viewed in cross section as shown in FIG. 2 and FIG. 3, and can also be folded in any other configuration without restraint.

While the balloon 33 is in a contracted state, the surface of the balloon 33 is smooth and flat due to the presence of the coating agent 33d.

In the balloon catheter 31, when the balloon 33 is expanded, the irregular portion of the surface of the base portion 33x protrudes through the surface of the coating agent 33d, and thereby the surface of the balloon 33 becomes irregular in the irregular region Q. Therefore, the balloon 33 can be even more easily fixed to an affected area.

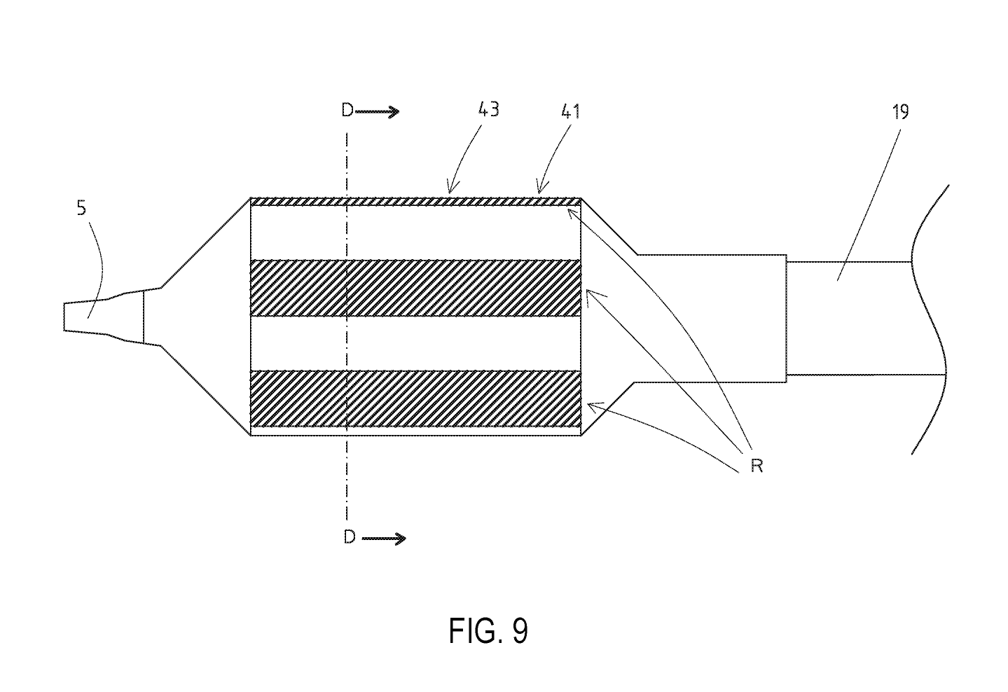

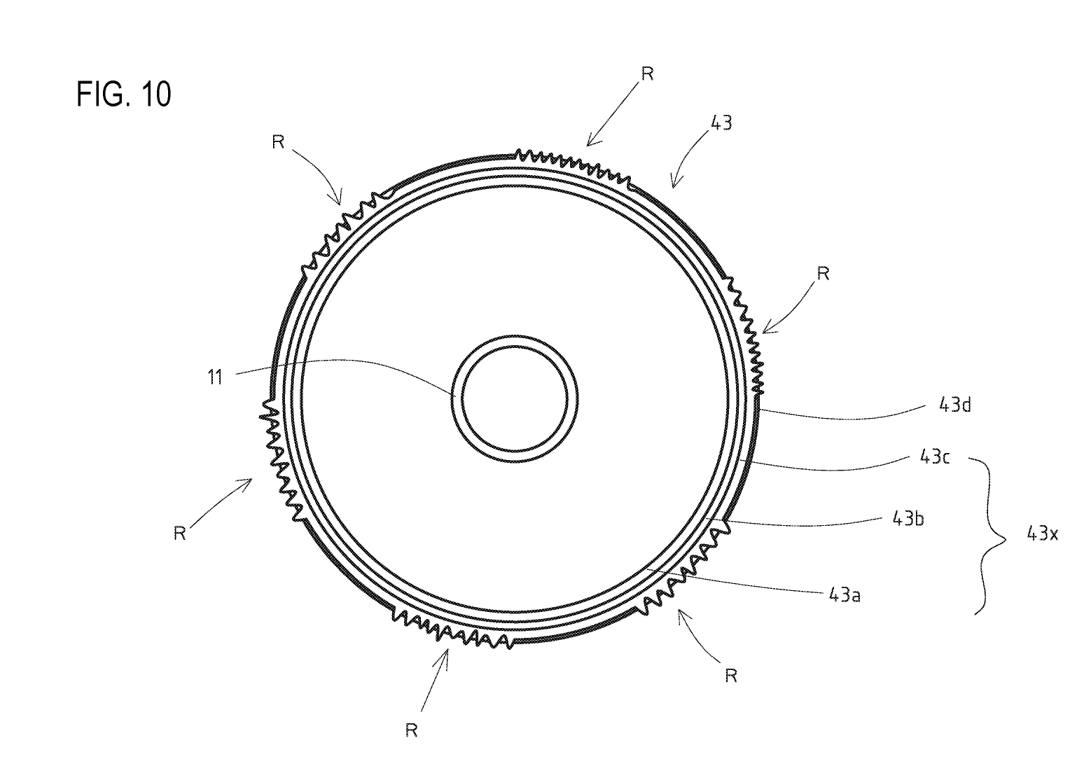

FIG. 9 illustrates an irregular region R of a balloon catheter 41 according to the disclosed embodiments (the balloon in the figure is in an expanded state), and FIG. 10 is a sectional view taken from line D-D of FIG. 9.

The following description only includes the differences from the balloon catheter 1.

Referring to FIG. 9, the balloon catheter 41 is also substantially the same as the balloon catheter 1 except for the balloon.

The irregular region R of a balloon 43 is different from the irregular region P of the balloon 3. More specifically, the irregular region P of the balloon 3 stretches over the entire circumference of the balloon 3 when viewed in cross section and stretches in the longitudinal direction of the balloon catheter 1 over the entire area where the balloon 3 has expanded to its maximum diameter. The irregular region R of the balloon 43 stretches over only parts of the circumference of the balloon 43 when viewed in cross section as shown in FIG. 10 (six regions) and stretches over the entire area where the balloon has expanded to its maximum diameter in the longitudinal direction of the balloon catheter 41 as shown in FIG. 9.

As discussed above, the balloon 3 comprises the base portion 3x including the irregular third base layer 3c overlaid on the smooth and flat surfaces of the first base layer 3a and the second base layer 3b, and the coating agent 3d coating the surface of the base portion 3x, with the following configuration: before the balloon 3 is expanded, the surface of the balloon 3 is smooth and flat due to the presence of the coating agent 3d; and when the balloon 3 is expanded, the irregular surface of the base portion 3x raises the coating agent 3d, and thereby the surface of the balloon becomes irregular in the irregular region P.

On the other hand, the balloon 43 comprises a base portion 43x constituted of an irregular third base layer 43c (with a partly irregular surface) overlaid on the smooth and flat surfaces of a first base layer 43a and a second base layer 43b, and a coating agent 43d coating the surface of the base portion 43x, with the following configuration: before the balloon 43 is expanded, the surface of the balloon 43 is smooth and flat due to the presence of the coating agent 43d; and when the balloon 43 is expanded, the irregular portion of the surface of the base portion 43x protrudes through the surface of the coating agent 43d, and thereby the surface of the balloon 43 becomes irregular in the irregular region R.

The balloon 43 in a contracted state is not directly described but is the same as the corresponding state for the balloon 3. The balloon 43 can be folded at four or six locations along its circumference when viewed in cross section as shown in FIG. 2 and FIG. 3, and can also be folded in any other configuration without restraint.

While the balloon 43 is in a contracted state, the surface of the balloon 43 is smooth and flat due to the presence of the coating agent 43d.

In the balloon catheter 41, when the balloon 43 is expanded, the irregular portion of the surface of the base portion 43x protrudes through the surface of the coating agent 43d, and thereby the surface of the balloon 43 becomes irregular in the irregular region R. Therefore, the balloon 43 can be even more easily fixed to an affected area.

What is claimed is:

1. A balloon catheter comprising:
a balloon comprising:
a base layer having a surface, at least a portion of the surface having an irregular texture; and
a coating layer coating the surface of the base layer,
wherein the balloon is configured so that:
when the balloon is not in an expanded state, an entire surface of the balloon is smooth, and
when the balloon is in an expanded state, at least a portion of the surface of the balloon has an irregular texture.

2. The balloon catheter according to claim 1, wherein the entire surface of the base layer has the irregular texture.

3. The balloon catheter according to claim 1, wherein only a portion of the surface of the base layer has the irregular texture.

4. The balloon catheter according to claim 3, wherein the portion of the base layer having the irregular texture extends along an entire circumference of the balloon when viewed in cross section from a longitudinal direction of the balloon catheter.

5. The balloon catheter according to claim 4, wherein the portion of the base layer having the irregular texture includes multiple regions that are spaced apart in the longitudinal direction.

6. The balloon catheter according to claim 3, wherein the portion of the base layer having the irregular texture extends along only a part of a circumference of the balloon when viewed in cross section from a longitudinal direction of the balloon catheter.

7. The balloon catheter according to claim 1, wherein the balloon is configured so that when the balloon is in the expanded state, the portion of the surface of the base layer that has the irregular texture protrudes through the coating layer.

8. The balloon catheter according to claim 2, wherein the balloon is configured so that when the balloon is in the expanded state, the portion of the surface of the base layer that has the irregular texture protrudes through the coating layer.

9. The balloon catheter according to claim 3, wherein the balloon is configured so that when the balloon is in the expanded state, the portion of the surface of the base layer that has the irregular texture protrudes through the coating layer.

10. The balloon catheter according to claim 4, wherein the balloon is configured so that when the balloon is in the expanded state, the portion of the surface of the base layer that has the irregular texture protrudes through the coating layer.
